(19) United States
(12) Patent Application Publication
(10) Pub. No.: US 2026/0148829 A1
(43) Pub. Date: May 28, 2026

(54) THERAPY DROPOUT SEVERITY PREDICTION USING MACHINE LEARNING

(71) Applicants: ResMed Pty Ltd.; ResMed Digital Health, Inc., San Diego, CA (US); ResMed Inc., San Diego, CA (US); ResMed Asia Pte. Ltd., Singapore (SG)

(72) Inventors: Chinh Duc NGUYEN, Sydney (AU); Hussein EMAMI, Singapore (SG); Philomena Schlexer LAMOUREUX, Encinitas, CA (US); Gregory Boyd REHM, San Diego, CA (US)

(21) Appl. No.: 19/496,851

(22) PCT Filed: Jun. 21, 2024
(86) PCT No.: PCT/US2024/035051

§ 371 Date: Feb. 9, 2026

Related U.S. Application Data
(60) Provisional application No. 63/509,998, filed on Jun. 23, 2023.

(51) Int. Cl. G16H 20/40

(52) U.S. Cl. CPC G16H 20/40

(57) ABSTRACT

Techniques for machine learning-based dropout severity prediction are provided. Usage information for a patient is accessed, the usage information indicating participation, by the patient, in a respiratory therapy. A dropout event with respect to the respiratory therapy is identified based on evaluating the usage information using a consolidated usage rule. In response to identifying the dropout event, a dropout severity prediction is generated based on the usage information and a trained machine learning model. In response to determining that the dropout severity prediction satisfies one or more criteria, one or more interventions for the patient are initiated.

### CROSS-REFERENCE TO RELATED APPLICATIONS

This Application claims the benefit of and priority to U.S. Provisional Ser. No. 63/509,998, filed on Jun. 23, 2023, the entire contents of which are incorporated herein by reference.

### INTRODUCTION

Aspects of the present disclosure relate to machine learning. More specifically, aspects of the present disclosure relate to training and using machine learning to predict therapy dropout severity.

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA) and Central Sleep Apnea (CSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using respiratory therapy systems.

Each respiratory therapy system generally has a respiratory therapy device connected to a user interface (e.g., a mask) via a conduit and optionally a connector. The user wears the user interface and is supplied a flow of pressurized air from the respiratory therapy device via the conduit. The user interface generally is a specific category and type of user interface for the user, such as direct or indirect connections for the category of user interface, and full face mask, a partial face mask, nasal mask, or nasal pillows for the type of user interface. In addition to the specific category and type, the user interface generally is a specific model made by a specific manufacturer, e.g., AirFit™ F20 manufactured by ResMed.

There are generally a variety of techniques and workflows that can be used to select specific types or models of equipment for specific patients. In some cases, the therapy system used for a given patient can impact user compliance with the therapy. For example, the specific user interface selected may affect compliance based on whether the user finds it comfortable to wear. A wide variety of other factors may similarly affect compliance, however, and predicting or identifying compliance can be difficult. A dropout event refers to when a patient stops engaging in therapy (or when their engagement or usage drops below defined thresholds or otherwise fails to meet defined criteria) for at least some period of time (which may be temporary or permanent). These events are harmful and can reduce or eliminate the benefits of therapy. However, predicting and evaluating such events remains difficult or impossible using conventional techniques.

Improved systems and techniques to predict dropout severity and thereby improve therapy compliance and outcomes are needed.

### SUMMARY

According to one embodiment presented in this disclosure, a method is provided. The method includes: accessing first usage information for a first patient, the first usage information indicating participation, by the first patient, in a respiratory therapy; identifying, based on evaluating the first usage information using a consolidated usage rule, a first dropout event with respect to the respiratory therapy; in response to identifying the first dropout event, generating a first dropout severity prediction based on the first usage information and a trained machine learning model; and in response to determining that the first dropout severity prediction satisfies one or more criteria, initiating one or more interventions for the first patient.

According to one embodiment of the present disclosure, a method is provided. The method includes: determining severity criteria indicating when dropout events for respiratory therapy are severe; accessing first usage information for a first patient, the first usage information indicating participation, by the first patient, in a respiratory therapy; in response to determining that the first usage information corresponds to a dropout event, labeling the first usage information based on the severity criteria; training a machine learning model to predict dropout severity based on the labeled first usage information; and deploying the machine learning model to generate dropout severity predictions.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example workflow to train dropout severity prediction machine learning models, according to one embodiment of the present disclosure.

FIG. 2 depicts an example workflow to use machine learning to predict dropout severity, according to one embodiment of the present disclosure.

FIG. 3 is a flow diagram depicting an example method for training dropout severity prediction machine learning models, according to one embodiment of the present disclosure.

FIG. 4 is a flow diagram depicting an example method for using machine learning to predict therapy dropout severity, according to one embodiment of the present disclosure.

FIG. 5 is a flow diagram depicting an example method for generating appropriate interventions based on dropout severity, according to one embodiment of the present disclosure.

FIG. 6 is a flow diagram depicting an example method for using machine learning to predict dropout severity, according to one embodiment of the present disclosure.

FIG. 7 is a flow diagram depicting an example method for training machine learning models to predict dropout severity, according to one embodiment of the present disclosure.

FIG. 8 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved therapy dropout prediction and dropout severity prediction.

Respiratory therapy (e.g., to treat obstructive sleep apnea (OSA)) often involves use of a therapy device (referred to in some aspects as a flow generator) to provide airflow to assist breathing. For example, a continuous positive airway pressure (CPAP) machine may be used to provide continuous positive pressure overnight while the user sleeps. Patient dropout (when a patient ceasing to be compliant with their therapy device usage) is a significant concern in respiratory therapy. Often, patients stop using their devices for relatively short periods of time before re-starting proper usage. For example, a patient may stop usage of their therapy device for a few weeks due to travel or vacation. In some embodiments, such short-term dropout (e.g., non-compliance that lasts for less than a defined threshold duration, such as a number of days or weeks) may be referred to as non-severe or non-critical dropout (e.g., because the patient will likely begin therapy again on their own, without prompting).

In some cases, patients may stop engaging in therapy for relatively long time periods (e.g., indefinitely or permanently). Causes for such long-term dropout vary, but can include concerns such as improper mask fit, discomfort, improper therapy settings (e.g., improper pressure settings), and the like. In some embodiments, such long-term dropout (e.g., non-compliance that lasts indefinitely or for more than a defined threshold duration, such as a number of days or weeks) may be referred to as severe or critical dropout (e.g., because the patient may be unlikely to begin therapy without intervention).

In some embodiments, machine learning model(s) can be trained and used to generate dropout severity predictions indicating the type of dropout a patient will have (or is having) based on their usage of their therapy device(s). For example, machine learning may be used to predict whether an active or future dropout event will be short (e.g., non-critical) or long (e.g., critical or severe). In some embodiments, the machine learning system leverages patient usage history and process it into time series data that can then be utilized for predictive purposes. In some embodiments, the models learn to predict long-term dropout, rather than short-term. That is, it may be difficult to accurately predict the probability that a dropout event is non-critical. However, experimentation has shown that models can be reliably trained to predict the probability that a dropout event is severe or long-term.

In some embodiments, dropout events can be detected using one or more defined adherence rules. Generally, each adherence rule specifies usage criteria that should be satisfied for a patient to be considered “compliant” with their respiratory therapy. Such usage criteria can generally include a variety of considerations and thresholds. For example, one adherence rule may specify a minimum total or average device usage (e.g., minimum number of hours) per day over a defined window (e.g., the last week). As another example, an adherence rule may specify a maximum gap in usage (e.g., a maximum number of sequential days without usage) over a defined period (e.g., the last 30 days, the last 90 days, and the like).

In some aspects, each adherence rule may correspond to a respective locale (e.g., which may be a physical locale, such as a specific region, country, and the like, as well as a jurisdictional or healthcare locale, such as when different healthcare providers or entities use different adherence rules to define compliance). In some aspects, to improve dropout detection, a consolidated rule can be defined based on a plurality of locale-specific adherence rules. For example, applying the consolidated rule may include evaluating whether a 30 day gap rule is satisfied, whether a 90 day gap rule is satisfied, whether a French long-term adherence (LTA) rule is satisfied, whether a German LTA rule is satisfied, whether a minimum average usage per day rule is satisfied, and the like.

In some aspects, patient data (e.g., usage information) can be monitored or evaluated periodically (e.g., daily) using the consolidated rule to determine whether a dropout event has begun, is about to begin, and/or is ongoing, as discussed in more detail below. If so, the system may use one or more machine learning models to predict whether the dropout event is (or will be) severe or critical (e.g., long-term) or non-severe or non-critical (e.g., short-term). Based on this prediction, various interventions may be triggered. For example, if the dropout is predicted to be severe, the system may initiate or facilitate interventions aimed at ending the patient's non-compliance and re-engaging in therapy. If the dropout is predicted to be temporary or non-severe (or is not predicted to be severe), the system may refrain from initiating interventions (e.g., to preserve computational and healthcare resources that need not be expended).

In this way, aspects of the present disclosure can significantly improve therapy compliance, which results in substantial improvements for individual patient outcomes. Further, by using trained models, embodiments of the present disclosure enable objective and accurate selective resource usage, which enables improved outcomes with reduced expense. For example, many interventions involve generation and/or transmission of digital content (e.g., multimedia content such as videos) which inherently consumes substantial computing resources to generate, store, transmit, and deliver (including memory and storage requirements, network bandwidth, electrical power consumed during such transmissions and output, and the like). Further, as some such content is delivered to mobile devices (e.g., the user's smartphone), refraining from delivering content that is not needed (e.g., because the dropout is not critical) can reduce battery consumption and resource usage on the user's device. Similarly, other interventions often involve manual effort or use of physical resources. For example, some interventions may include a user (e.g., a doctor or other healthcare provider) calling or otherwise contacting the patient to discuss the non-compliance, or sending physical resources (e.g., mailing pamphlets or other content) to the patient. By selectively using such interventions using machine learning, the resources consumed can be substantially reduced.

Moreover, in some embodiments, dropout severity is only predicted (using machine learning) if a dropout is detected by the consolidated rule. That is, the model may be selectively run only when needed, rather than continuously (e.g., only when dropout is detected, rather than daily). This targeted execution of the machine learning model can reduce computational burden on the system, as compared to more conventional approaches that use the model repeatedly (e.g., daily for all users).

### Example Workflow to Train Compliance Prediction Machine Learning Models

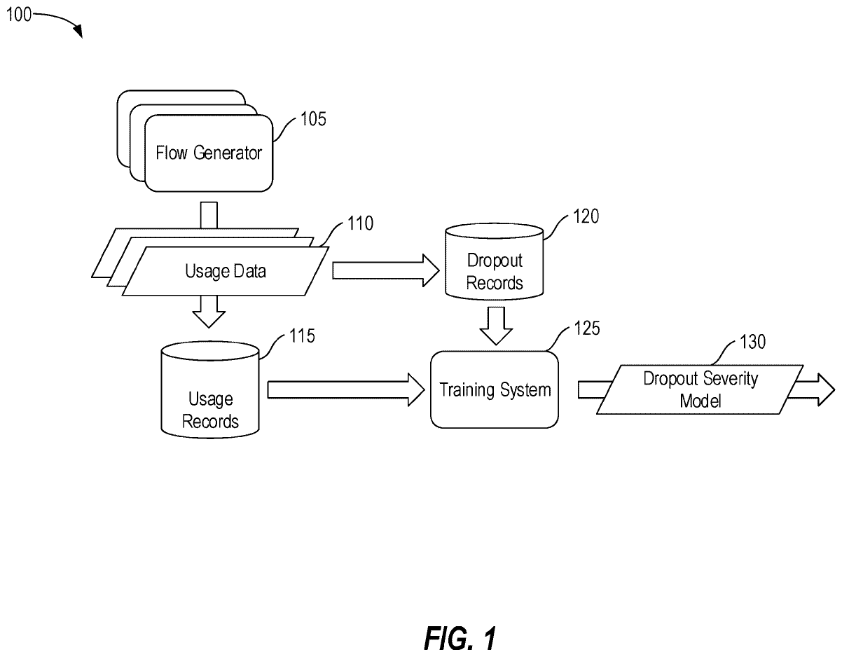

FIG. 1 depicts an example workflow 100 to train dropout severity prediction machine learning models, according to one embodiment of the present disclosure.

In the illustrated example, usage data 110 is accessed from one or more flow generators 105 and stored as usage records 115. As used herein, “accessing” data may generally include receiving, retrieving, collecting, generating, requesting, obtaining, or otherwise gaining access to the data. For example, the flow generators 105 may collect usage data while in use (e.g., duration of use), and transmit the usage data 110 to a repository for usage records 115 via one or more networks (which may include the Internet). The flow generators 105 are generally representative of respiratory therapy devices (e.g., CPAPs, bi-level positive airway pressure (BiPAP) machines, and the like) used to provide respiratory therapy for patients. Generally, usage data 110 for any number and variety of patients may be collected to train the model(s). In some embodiments, the usage data 110 is referred to as historical or training usage data.

The usage data 110 may generally be representative of a wide variety of usage information for the respiratory therapy. For example, the usage information may indicate participation in a therapy. In some embodiments, each record of usage data 110 indicates the usage duration of a user over one or more days. For example, the usage data 110 may indicate, for a first patient, one or more lengths of time (e.g., a number of minutes or hours) that the first patient wore a user interface (e.g., a CPAP mask) for their respiratory therapy over one or more days (e.g., over the last seven days). This may include, for example, the usage duration per day, and/or the total, minimum, maximum, and/or average duration of use during the window. In some embodiments, the usage data 110 includes usage information for the prior day or night (e.g., for the last usage session), and the usage records 115 accumulate the usage over one or more windows of time (e.g., to indicate average usage in rolling or sliding seven-day windows).

In some aspects, the usage data 110 may additionally include other patient information (or additional patient information may be accessed from other sources). For example, in some aspects, the additional patient information may include more information relating to the patient and/or the therapy, such as the patient's apnea-hypopnea index (AHI) (including the AHI of the last usage session, the average, maximum, and/or minimum AHI over a window, and the like), the patient's hypopnea index (HI) (including the HI of the last usage session, the average, maximum, and/or minimum HI over a window, and the like), the number of times that the patient donned and doffed their user interface (e.g., put on and took off their mask) while using their flow generator 105 (including the number of times during the last usage session, the average, maximum, and/or minimum number of times per session over a window, and the like), the patient's respiratory effort related arousal (RERA) index (including the RERA of the last usage session, the average, maximum, and/or minimum RERA over a window, and the like), and the like.

In the illustrated example, the usage data 110 is further used to define or generate a set of dropout records 120. Although the illustrated example depicts use of the usage data 110 directly to create compliance records, in some aspects, the dropout records 120 may be generated based at least in part on the usage records 115. Each dropout record 120 may generally correspond to a dropout event for a given patient at a given point in time. For example, the usage data 110 and/or usage records 115 may be evaluated (e.g., by the training system 125) to identify dropout events and generate corresponding dropout records 120. As discussed above, each dropout event generally corresponds to a time when a patient became non-compliant with respiratory therapy (as determined by evaluating the usage data 110 of the patient using a consolidated rule, for example).

For example, in some embodiments, the consolidated rule may be defined based on a plurality of adherence rules, and the consolidated rule may indicate that a dropout has begun whenever one or more of the plurality of adherence rules are not satisfied. In some embodiments, the consolidated rule uses a majority voting approach, where the patient is deemed compliant if a majority of the adherence rules indicate compliance (or where the patient is deemed non-compliant if a majority of the adherence rules indicate non-compliance). In at least one embodiment, once the system determines that the consolidated rule is not satisfied (e.g., the patient is deemed non-compliant), the system may determine when the dropout event occurred or began based on the plurality of adherence rules. For example, when the consolidated rule is violated, the system may identify the set of adherence rules that currently indicate non-compliance, and determine which of these rules was violated first (e.g., which violated adherence rule first indicated non-compliance). This earliest time may then be used as the start or begin time for the dropout event.

That is, the usage data 110 and/or usage records 115 may be evaluated periodically (e.g., daily) to determine whether any adherence rules are violated. When a sufficient combination of adherence rules are violated such that the consolidated rule is violated, the system may, for each currently violated adherence rule, trace back in time until the rule was not violated. The earliest of these times is then used as the time or date of occurrence (or beginning of occurrence) for the dropout.

In some aspects, each dropout record 120 may further indicate the duration of the dropout. For example, if the dropout has ended (e.g., the user became compliant again, as determined using the consolidated rule), the dropout record 120 may indicate when the dropout began and how long it lasted. If the dropout has not ended (e.g., the user is still non-compliant, as determined using the consolidated rule), the dropout record 120 may indicate when the dropout began and the current (running) duration. In some aspects, rather than indicating the duration of the dropout, the dropout records 120 may each indicate whether the corresponding dropout was critical (e.g., it lasted longer than a defined threshold) or non-critical (e.g., it was shorter than the threshold).

In some aspects, the severity or criticality threshold may be defined based on evaluation of historical compliance data for respiratory therapy patients. For example, by evaluating a relatively large number of prior or current patients, the system (or a user) may be able to identify dropout patterns. As one example, after some number of days or weeks from when the user enters non-compliance, it may be determined (based on the historical data) that they are unlikely to begin therapy again without intervention. This duration may then be used as the threshold to define long-term and short-term (also referred to as critical or severe and non-critical or non-severe) dropout.

In the illustrated workflow 100, a training system 125 uses the usage records 115 and dropout records 120 to train one or more dropout severity models 130. The dropout severity model 130 generally corresponds to a machine learning model that predicts the severity of a therapy dropout event (e.g., the duration). Generally, the training system 125 may use a variety of architectures for the dropout severity model 130 depending on the particular implementation. For example, the dropout severity model 130 may be a light gradient boosting machine (LightGBM) model, a logistic regression model, a Gaussian Baive Bayes model, a random forest, an extreme gradient boosting (XGBoost) model, and the like. Additionally, although illustrated as a discrete system for conceptual clarity, in some aspects, the training system 125 may be implemented using hardware, software, or a combination of hardware and software, and may be implemented as a component of a broader system and/or may be distributed across multiple systems.

In some aspects, the usage records 115 and dropout records 120 are used to form training records or exemplars, where each exemplar corresponds to a dropout (reflected in the dropout records 120). Each exemplar may include a set of input data (e.g., usage records 115 for one or more windows or points in time prior to the dropout event beginning) and a set of label or target output data (e.g., the duration of the corresponding dropout event, and/or a binary indication as to whether the dropout event was classified as long-term or critical). Generally, the format of the label data may vary depending on the preferred output. For example, if the training system 125 is training the dropout severity model 130 to predict dropout duration directly (e.g., a regression task), the label may include the duration of the historical dropout event. If the training system 125 is training the dropout severity model 130 to predict whether a dropout is severe or critical (e.g., whether it will last longer than a defined threshold), the label may indicate whether the historical dropout was severe (e.g., whether it lasted longer than the threshold).

In some embodiments, the content and scope of the input data in each training exemplar may vary depending on the particular implementation. For example, in some embodiments, each exemplar may include usage records 115 from a defined window or period of time (e.g., 90 days prior to the beginning of the dropout event). In some embodiments, as discussed above, the input data may further include one or more additional elements, such as the patient's AHI, HI, RERA, mask donning and doffing cycles, and the like.

In some embodiments, rather than applying these inputs directly to the model, the training system 125 (or another system) may perform preprocessing and/or feature extraction on some or all of the input data. For example, in one embodiment, the training system 125 may use preprocessing to fill in missing values (e.g., imputing a value of zero or negative one for any dates without reported usage data), apply min-max scaling or normalization (e.g., scaling the values to between zero and one), and the like. In some embodiments, the training system 125 may perform feature extraction on some or all of the inputs. For example, from the time series of usage data (e.g., indicating duration of usage for each day in a sequence of days), one or more relevant features may be generated or extracted, such as linear and non-linear autocorrelations, successive differences, value distributions and outliers, and the like. In some embodiments, similar features (such as minimum, maximum, and average values) may be extracted for additional inputs (if any), such as the AHI, HI, RERA, and the like. These extracted features may then be used as input to the model.

In this way, the training system 125 uses the usage records 115 and dropout records 120 to train the dropout severity model 130 to predict dropout severity. Generally, the specific techniques used to train the dropout severity model 130 may vary depending on the particular implementation and architecture of the model.

In the illustrated example, after the dropout severity model 130 is trained, it may be deployed for inferencing by one or more systems. In some embodiments, the training system 125 may itself deploy the model for inferencing locally. That is, the training system 125 may instantiate an instance of the dropout severity model 130 locally, and use it to predict the severity of detected dropouts based on new usage data 110. In some embodiments, the model may be deployed to one or more inferencing systems (separate from the training system 125), as discussed in more detail below.

### Example Workflow to Use Machine Learning to Predict Therapy Dropout Severity

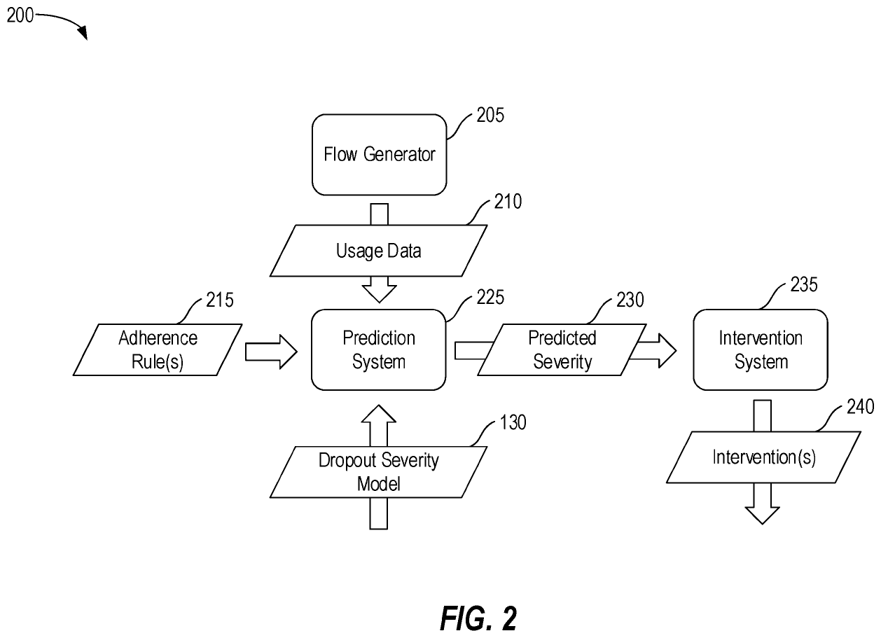

FIG. 2 depicts an example workflow 200 to use machine learning to predict dropout severity, according to one embodiment of the present disclosure.

In the illustrated example, a prediction system 225 accesses one or more adherence rules 215, usage data 210 from one or more flow generators 205, and a trained dropout severity model 130 and generates predicted severity 230 for one or more dropout events. Although illustrated as a discrete system for conceptual clarity, in some aspects, the prediction system 225 may be implemented using hardware, software, or a combination of hardware and software, and may be implemented as a component of a broader system and/or may be distributed across multiple systems.

In some embodiments, as discussed above, the flow generator 205 may generally collect usage data while in use (e.g., duration of use), and transmit the usage data 210 to the prediction system 225 (or to a repository for usage data, which the prediction system 225 may access) via one or more networks (which may include the Internet). For example, the usage data 210 from each flow generator 205 may be stored in a repository and evaluated by the prediction system 225 periodically (e.g., daily) or in response to other criteria. The flow generator 205 is generally representative of any respiratory therapy device (e.g., CPAPs, BiPAPs, and the like) used to provide respiratory therapy for patients.

The usage data 210 may generally be representative of a wide variety of usage information for the respiratory therapy. In some embodiments, each record of usage data 210 indicates the usage duration of a user over one or more days. For example, the usage data 210 may indicate, for a given patient, one or more lengths of time (e.g., a number of minutes or hours) that the first patient wore a user interface (e.g., a CPAP mask) for their respiratory therapy over one or more days (e.g., over the last seven days). This may include, for example, the usage duration per day, and/or the total, minimum, maximum, and/or average duration of use during the window. In some embodiments, the usage data 210 includes usage information for the prior day or night (e.g., for the last usage session), and the prediction system 225 (or another system) may accumulate the usage over one or more windows of time (e.g., to indicate average usage in rolling or sliding seven-day windows).

In some aspects, the usage data 210 may additionally include other patient information (or additional patient information may be accessed from other sources). For example, in some aspects, the additional patient information may include more information relating to the patient and/or the therapy, such as the patient's AHI (including the AHI of the last usage session, the average, maximum, and/or minimum AHI over a window, and the like), the patient's HI (including the HI of the last usage session, the average, maximum, and/or minimum HI over a window, and the like), the number of times that the patient donned and doffed their user interface (e.g., put on and took off their mask) while using their flow generator 205 (including the number of times during the last usage session, the average, maximum, and/or minimum number of times per session over a window, and the like), the patient's RERA index (including the RERA of the last usage session, the average, maximum, and/or minimum RERA over a window, and the like), and the like.

In some embodiments, as discussed above, the adherence rule(s) 215 each generally indicate a set of criteria that can be used to evaluate usage data 210 to determine whether the rule is satisfied (e.g., whether the patient is compliant with the therapy). In some embodiments, each adherence rule 215 has a different set of criteria, and may correspond to a different locale (e.g., different regions using different rules, different healthcare providers using different rules, and the like). The specific criteria and thresholds used by each adherence rule 215 may vary, but each generally includes evaluation of usage data such as the minimum or average usage durations, a maximum number of days with zero usage, and the like.

In some embodiments, the prediction system 225 (or another system) defines a consolidated rule based on a combination of adherence rules 215. For example, in some embodiments, determining whether the consolidated rule is satisfied comprises determining whether at least a defined number or proportion of the adherence rules 215 are satisfied (e.g., at least one, at least one third, at least a majority, all of the rules, and the like). That is, in one embodiment, the prediction system 225 may determine that the consolidated rule is not satisfied if a majority of the adherence rules 215 are not satisfied.

In some aspects, as discussed above, the prediction system 225 may determine that the consolidated rule was not satisfied and/or that a dropout event began retroactively or in the past. For example, suppose there are three adherence rules 215: Rule A, Rule B, and Rule C. Suppose further that on day one, all three rules were satisfied (e.g., the patient was compliant). Suppose further that on day two, Rule A was violated but Rules B and C were satisfied. If a majority-rule approach is used for the consolidated rule, the prediction system 225 will determine, on day two, that the consolidated rules is satisfied. Suppose further that on day three, Rules A and B are violated, but Rule C is satisfied. In an embodiment, the prediction system 225 may determine (on day three) that the consolidated rule is violated. In some embodiments, the prediction system 225 may then evaluate the historical determinations and find that the earliest rule violation occurred on day one (when Rule A was violated). Therefore, the prediction system 225 may determine that the dropout event occurred or began on day one, even though the consolidated rule was (at the time) satisfied.

In the illustrated example, the prediction system 225 accesses the dropout severity model 130 to evaluate usage data 210. Although depicted as receiving the model from an external source (e.g., from the training system 125 of FIG. 1), in some embodiments, the prediction system 225 may alternatively train or access the model locally. In some embodiments, the prediction system 225 may access the dropout severity model 130 by receiving the trained model (e.g., the learned parameters) from a training system, allowing the prediction system 225 to instantiate the model locally for inferencing. In some embodiments, the prediction system 225 may access the model by transmitting queries (e.g., usage data 210 or features extracted therefrom) to another system that provides the model for inferencing (e.g., via one or more application programming interfaces (APIs).

In some embodiments, as discussed above, the prediction system 225 may evaluate the usage data 210 received from each flow generator 205 (e.g., for each user) using the consolidated rule (e.g., one or more adherence rules 215) to identify dropout events (e.g., to identify patients that are beginning, recently began, or are about to begin a dropout period). For example, the prediction system 225 may evaluate the usage data 210 periodically (e.g., daily) and/or whenever new usage data 210 is available.

In the illustrated example, for any identified dropout events, the prediction system 225 may access corresponding usage information for the corresponding patient, and process it using the dropout severity model 130 to generate a corresponding predicted severity 230. In some embodiments, as discussed above, the prediction system 225 may perform one or more preprocessing and/or feature extraction steps, and provide these processed features as model input.

In some embodiments, the prediction system 225 uses the current usage data 210 (e.g., for the current day) as input to generate the predicted severity 230. In some embodiments, the prediction system 225 accesses historical usage data (e.g., data from the day before the dropout event began) and uses this data as input. In some embodiments, the prediction system 225 accesses a time sequence or set of historical data (e.g., usage data from an N day window that ends on (or just before) the day when the dropout event began).

In some embodiments, as discussed above, the prediction system 225 may additionally or alternatively access other patient information, such as their AHI, HI, RERA, mask don/doff information, and the like. These additional data points may also be used as input for the model.

As discussed above, the particular format and context of the predicted severity 230 may vary depending on the particular implementation. In some embodiments, the predicted severity 230 comprises a binary classification indicating whether the current dropout is predicted to be severe or critical (e.g., whether it is predicted to last beyond a defined duration). In some embodiments, the predicted severity 230 additionally or alternatively includes a confidence or probability that the dropout is severe or critical.

In the illustrated workflow 200, the predicted severity 230 is accessed by an intervention system 235. Although illustrated as a discrete system for conceptual clarity, in some aspects, the intervention system 235 may be implemented using hardware, software, or a combination of hardware and software, and may be implemented as a component of a broader system and/or may be distributed across multiple systems. For example, the intervention system 235 may be implemented as a component of the prediction system 225 (or both the prediction system 225 and the intervention system 235 may be components of a broader system).

In some embodiments, as discussed above, the intervention system 235 may evaluate the predicted severity 230 for each patient that is currently experiencing a dropout event in order to determine which, if any, interventions 240 should be provided. For example, in some embodiments, the intervention system 235 may determine that if the predicted severity 230 does not satisfy one or more criteria (e.g., if the predicted severity 230 indicates that the dropout is not predicted to be critical, and/or of the probability or confidence that it is critical does not meet or exceed one or more thresholds), the intervention system 235 may refrain from providing any interventions 240 for the corresponding patient. For example, the intervention system 235 may infer that the dropout is temporary and/or that the patient is likely to restart therapy on their own. In some embodiments, even if the dropout is predicted to be non-critical, the intervention system 235 may nevertheless provide some (relatively less intrusive or expensive) intervention 240, as compared to if the dropout is severe.

In some embodiments, if the predicted severity 230 satisfies one or more criteria (e.g., the dropout is predicted to be severe, critical, and/or long-term), the intervention system 235 can initiate one or more interventions 240 for the patient. As used herein, initiating interventions 240 may generally include performing the intervention 240 directly (e.g., transmitting a message), triggering or instructing another system or user to perform the intervention, and the like.

Generally, the interventions 240 may vary depending on the particular implementation. For example, in some embodiments, the interventions 240 may include transmitting one or more messages to the patient (e.g., via text, direct message, in-application messaging, email, and the like), providing multimedia content (e.g., sending or providing a link to a video, images and text, or other multimedia), notifying one or more healthcare providers or other users associated with the patient (e.g., notifying the patient's doctor of the critical dropout), and the like. In some embodiments, the intervention system 235 can select the intervention based at least in part on the predicted criticality of the dropout. For example, if the dropout is predicted to be severe with a high degree of confidence, the intervention system 235 may initiate more costly or invasive interventions than if the confidence is low.

Generally, although the particular techniques used to select and provide particular interventions 240 for particular patients may vary, the general goal of the interventions 240 is to bring the patient back into compliance, such as by providing tips, asking questions about why the patient is non-compliant, and the like.

### Example Method for Training Therapy Dropout Severity Prediction Machine Learning Models

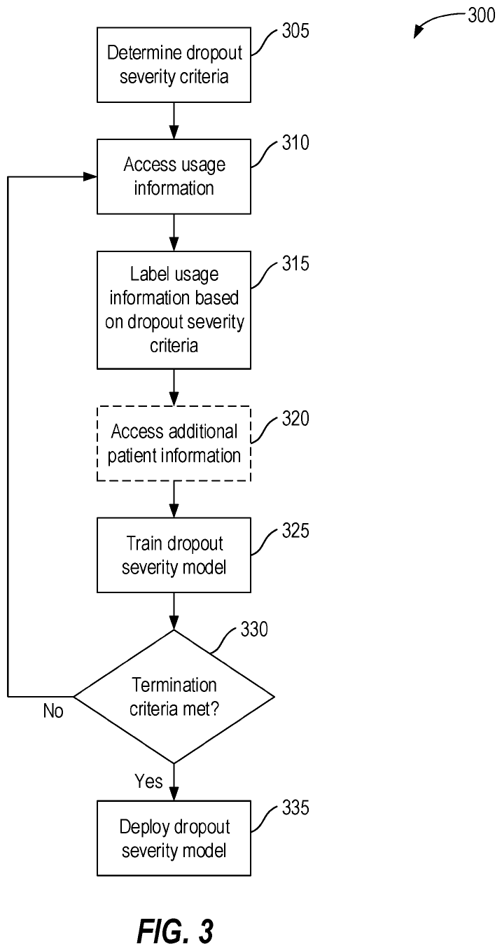

FIG. 3 is a flow diagram depicting an example method 300 for training dropout severity prediction machine learning models, according to one embodiment of the present disclosure. In some embodiments, the method 300 is performed by a training system, such as the training system 125 of FIG. 1. In some aspects, the method 300 provides additional detail for the workflow 100 of FIG. 1.

At block 305, the training system determines one or more dropout severity criteria. In some embodiments, as discussed above, the dropout severity criteria may be used to determine whether a dropout event is severe. For example, the dropout severity criteria may indicate that dropouts that are shorter than a defined duration are non-critical or non-severe, and/or that dropouts lasting longer than the defined duration are critical or severe. In some embodiments, the dropout severity criteria is manually defined or curated (e.g., by a data scientist after reviewing historical data). In some embodiments, the dropout severity criteria may be automatically or programmatically determined (e.g., by using one or more evaluation techniques or operations to evaluate the historical data).

At block 310, the training system accesses a set of usage information for training a machine learning model (e.g., a dropout severity model, such as the dropout severity model 130 of FIG. 1). For example, the training system may access data such as usage records 115 of FIG. 1. As discussed above, the usage records may generally include, for each patient of a set of patients usage information from the user (e.g., lengths of time that the user used or engaged in the therapy for one or more days). For example, as discussed above, the usage information may indicate, for each day of a sequence of days, the number of minutes or hours that the patient used their therapy equipment (or otherwise engaged in the therapy). In some aspects, the usage information corresponds to a patient that experienced a dropout event. For example, the usage information may include usage data covering a defined window (e.g., thirty days) prior to the dropout beginning.

At block 315, the training system labels the usage information (accessed at block 310) based on the dropout severity criteria (at block 305). For example, as discussed above, the training system may determine whether the dropout event (e.g., the dropout that followed the usage information) had a duration that satisfies the dropout severity criteria (e.g., whether it was longer than the threshold duration). If so, the training system may label the usage information to indicate that it preceded a severe dropout. If not, the training system may label the usage information to indicate that it did not precede a severe dropout. In some aspects, as discussed above, this may include labeling it as preceding a non-severe dropout.

At block 320, the training system optionally accesses additional patient information for the patient. For example, as discussed above, the training system may access additional data corresponding to the defined window (prior to the dropout event), such as their AHI from one or more days, HI from one or more days, RERA index from one or more days, number of interface donning and doffing actions for one or more days, and the like.

Although not depicted in the illustrated example, in some aspects, the training system optionally preprocesses the usage information and/or additional information to prepare it for input to a machine learning model. For example, depending on the particular implementation, the training system may generate or extract feature information. As one example, the training system may extract time series features from the usage information, extract or determine features such as the maximum, minimum, average, and/or median values for one or more additional features, and the like.

At block 325, the training system trains one or more machine learning models based on the selected diagnostic record. Generally, the particular techniques used to train the model(s) may vary depending on the particular architecture and implementation. For example, in some aspects, the training system may process the diagnostic data using a machine learning model to generate an output prediction (e.g., a binary classification of predicted compliance and/or a probability of compliance). The training system may then generate a loss based on the predicted compliance and the ground truth compliance (determined at block 320), and refine the model parameters (e.g., weights and/or biases) based on the loss using gradient descent.

In some embodiments, as discussed above, the training system may train multiple machine learning models (e.g., one for each locale, and/or one for each of a variety of compliance criteria). In some such embodiments, at block 320, the training system may determine multiple compliance labels based on the variety of locales or compliance rules, as discussed above.

At block 330, the training system determines whether one or more termination criteria are met. Generally, evaluating the termination criteria can include a wide variety of operations, such as determining whether additional training data remains, determining whether additional epochs or rounds of training remain, determining whether the model has reached a desired accuracy, determining whether a defined amount of time or computational resources have been spent training the model, and the like.

If, at block 330, the training system determines that the criteria are not met, the method 300 returns to block 310 to access another set of usage information. If the training system determines that the criteria are met, the method 300 continues to block 335. Although the illustrated example depicts the training system refining the model based on each exemplar individually (e.g., using stochastic gradient descent) for conceptual clarity, in some aspects, the training system may additionally or alternatively refine the model using a set of diagnostic records (e.g., using batch gradient descent).

At block 335, the training system deploys the machine learning model to one or more inferencing systems. As discussed above, deploying the model may include a variety of operations depending on the particular implementation, including initiating the trained model locally for inferencing, transmitting the model artefacts to one or more dedicated inferencing systems (separate from the training system), and the like.

### Example Method for Using Machine Learning to Predict Therapy Dropout Severity

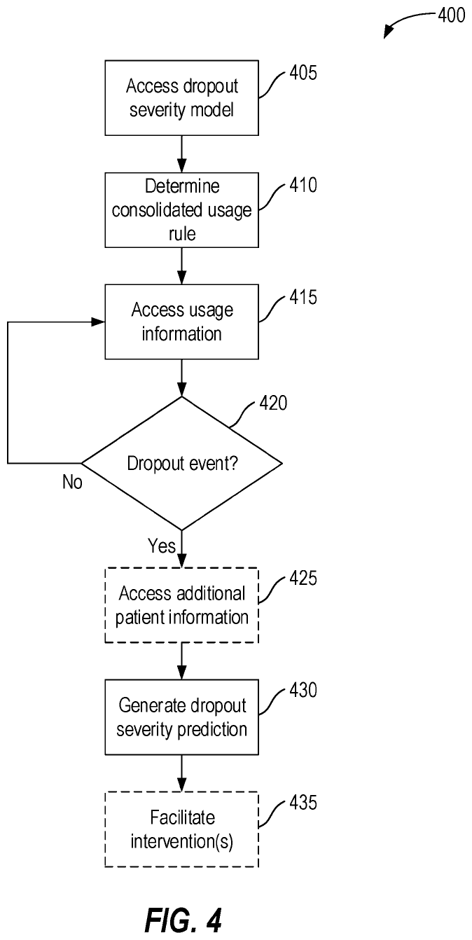

FIG. 4 is a flow diagram depicting an example method for using machine learning to predict therapy dropout severity, according to one embodiment of the present disclosure. In some embodiments, the method 400 is performed by a prediction system and/or an intervention system (which may be collectively referred to as an inferencing system), such as the prediction system 225 and/or intervention system 235, each of FIG. 2. In some aspects, the method 400 provides additional detail for the workflow 200 of FIG. 2.

At block 405, the inferencing system accesses a trained dropout severity model (e.g., trained using the method 300 of FIG. 3). As discussed above, accessing the trained model may generally include receiving or determining the parameters of the trained model and deploying it locally, accessing it via one or more API calls, and the like.

At block 410, the inferencing system determines a consolidated usage rule. As discussed above, the consolidated usage rule generally corresponds to a combination of one or more locale-specific adherence rules, and can be used to determine whether a patient is compliant with their respiratory therapy. In some embodiments, the consolidated rule (e.g., the particular combination) is defined manually (e.g., by a data scientist). For example, a user may specify that the consolidated rule includes evaluation of a defined set of rules, and that the consolidated rule is satisfied if a majority of the set of rules are satisfied. In some embodiments, the consolidated rule may be generated programmatically. For example, the inferencing system may evaluate the accuracy or sensitivity of each of the adherence rule, and generate an ensemble of rules (e.g., using learned weightings) to use as the consolidated rule.

At block 415, the inferencing system accesses usage information for a patient. For example, the inferencing system may access data such as usage data 210 of FIG. 2. As discussed above, the usage information may generally include, for a given patient, usage information for a therapy (e.g., lengths of time that the user used or engaged in the therapy for one or more days). For example, as discussed above, the usage information may indicate, for each day of a sequence of days, the number of minutes or hours that the patient used their therapy equipment (or otherwise engaged in the therapy). In some aspects, the inferencing system accesses the usage information for each patient periodically (e.g., daily).

At block 420, the inferencing system determines whether a dropout event is occurring or has begun (or is about to begin, depending on the particular implementation). For example, as discussed above, the inferencing system may evaluate the newly received usage information using the consolidated rule to determine whether it is satisfied. In some embodiments, as discussed above, application of the consolidated rule may include evaluating the usage information using each individual adherence rule, and determining whether the consolidated criteria are met (e.g., whether at least a subset of the adherence rules, such as a defined combination of adherence rules, are not satisfied, such as at least one, at least a majority, and the like).

If, at block 420, the inferencing system determines that a dropout event has not begun (e.g., the consolidated rule is satisfied), the method 400 returns to block 415 to access new usage data (either new data for the same patient, or new data for another patient). If, at block 420, the inferencing system determines that a dropout event has begun or is ongoing, the method 400 continues to block 425.

At block 425, the inferencing system optionally accesses additional patient information. For example, as discussed above, the inferencing system may determine the patient's AHI, HI, RERA, mask don/doff cycles, and the like for one or more days or windows of time prior to the dropout event. In some embodiments, as discussed above, the inferencing system may determine these additional inputs (and the usage information) for a defined window of time leading up to the beginning of the dropout, such as the past sixty days.

At block 430, the inferencing system generates a dropout severity prediction based on the usage information and/or additional information. For example, as discussed above, the inferencing system may process the data using the dropout severity model, as discussed above. In some embodiments, as discussed above, the inferencing system may optionally apply one or more preprocessing operations and/or feature extraction operations, such as assigning missing values with a defined value (e.g., zero or −1), applying scaling operations, extracting features, and the like.

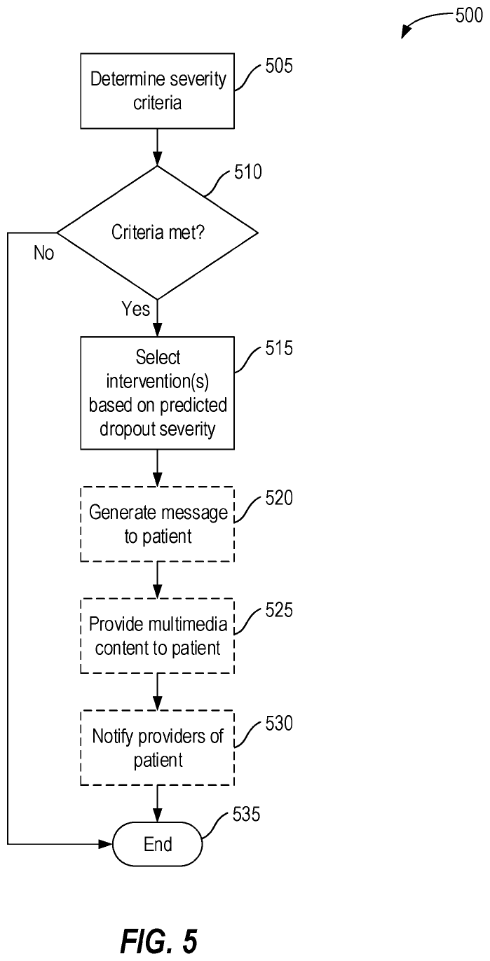

At block 435, the inferencing system optionally facilitates or initiates one or more interventions for the patient, depending on the dropout severity prediction. For example, as discussed above, if the dropout is predicted to be severe, the inferencing system may select interventions such as messaging, additional human guidance, and the like. In some embodiments, if the dropout is not predicted to be severe, the inferencing system may refrain from initiating such interventions. One example method for facilitating interventions is discussed in more detail below with reference to FIG. 5.

### Example Method for Generating Interventions Based on Dropout Severity

FIG. 5 is a flow diagram depicting an example method 500 for generating appropriate interventions based on dropout severity, according to one embodiment of the present disclosure. In some embodiments, the method 500 is performed by a prediction system and/or an intervention system (which may be collectively referred to as an inferencing system), such as the prediction system 225 and/or intervention system 235, each of FIG. 2. In some aspects, the method 400 provides additional detail for the workflow 200 of FIG. 2. In some aspects, the method 400 provides additional detail for block 435 of FIG. 4.

At block 505, the inferencing system determines one or more severity criteria. The severity criteria generally relate to whether the dropout severity prediction for a user warrants intervention, as discussed above. In some embodiments, for example, the severity criteria may indicate that intervention is justified or should be used if the dropout severity prediction indicates that the dropout is predicted to be severe (e.g., that it is predicted to last longer than a threshold duration). In some embodiments, as discussed above, the severity criteria may additionally or alternatively indicate one or more thresholds (e.g., if the severity prediction is a continuous value, rather than a classification) that, if exceeded, indicate that intervention should be used. In some embodiments, as discussed above, the criteria may additionally or alternatively relate to the confidence or probability of the prediction (e.g., indicating that interventions should be used if the dropout is predicted to be severe with high confidence, but that interventions should be withheld (or less intrusive and/or computationally expensive interventions should be used) if the confidence is lower.

In some embodiments, the severity criteria are defined manually (e.g., by an administrator). In some embodiments, the severity criteria can be defined programmatically, and/or may be applied differently depending on the implementation. For example, in some embodiments, the criteria may indicate that the top N (or the top M%) of patients having the highest predicted severity (or the highest confidence that the dropout is severe) should receive intervention. In at least some embodiments, the inferencing system may determine the available resources, and generate dynamic severity criteria based on these resources (e.g., to avoid oversubscribing the available resources, which may reduce efficacy for other users).

At block 510, the inferencing system determines whether the severity criteria are met with respect to a given patient. For example, as discussed above, the inferencing system may compare the patient's predicted dropout severity to the severity criteria. If the criteria are not met, the method 500 terminates at block 535. That is, the inferencing system refrains from providing targeted interventions for the patient, as their dropout is not expected to be severe (or the probability or confidence that it is severe is below a threshold). If, at block 510, the inferencing system determines that the criteria are met, the method 500 continues to block 515.

At block 515, the inferencing system selects one or more interventions based on the predicted dropout severity. For example, in some aspects, the inferencing system may select more aggressive interventions (e.g., interventions that consume more computational and/or physical resources, interventions that are more intrusive or require more manual effort, and the like) for dropouts that are predicted to be particularly severe (e.g., with a high severity indicating potential for increased harm to the patient, and/or dropouts that are predicted to be severe with high confidence). In some such embodiments, the inferencing system may select relatively less intrusive or expensive interventions for dropouts that are less severe (or where the inferencing system is less confident that the dropout is severe).

At block 520, the inferencing system optionally generates one or more messages or transmissions for the patient. For example, as discussed above, the inferencing system may transmit (or cause to be transmitted) an email, text message, application message, and the like. Generally, the particular contents of this message may vary depending on the particular implementation. For example, in some embodiments, the message may include encouragement, coaching (e.g., tips or suggestions to improve therapy), queries (e.g., asking how therapy is going and/or if the patient has any concerns or questions), and the like.

At block 525, the inferencing system optionally provides multimedia content to the patient. For example, as discussed above, the inferencing system may transmit (or cause to be transmitted) video content, image content, text content combined with video and/or image content, audio content, and the like. Generally, the particular contents of this multimedia may vary, as discussed above with respect to the messaging content. For example, in some embodiments, the multimedia may include encouragement, coaching (e.g., tips or suggestions to improve therapy), queries (e.g., asking how therapy is going and/or if the patient has any concerns or questions), and the like. In some aspects, the multimedia content may generally incur additional computational expense to deliver, as compared to messaging content.

At block 530, the inferencing system optionally notifies one or more providers associated with the patient. For example, as discussed above, the inferencing system may identify healthcare providers or other users with a defined relationship to the patient (e.g., caregivers, family members, and the like), and notify these user(s) that the patient is at risk of a critical dropout (e.g., via messaging, phone call, and the like). This may allow such other users to intervene, but may involve additional intrusion or difficulty, as compared to other interventions.

After providing any selected interventions, the method 500 terminates at block 535.

### Example Method for Using Machine Learning Models to Predict Dropout Severity

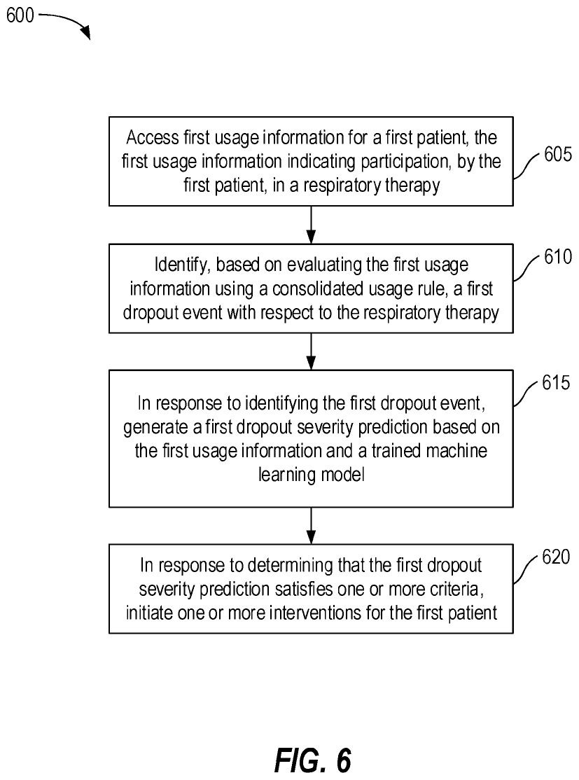

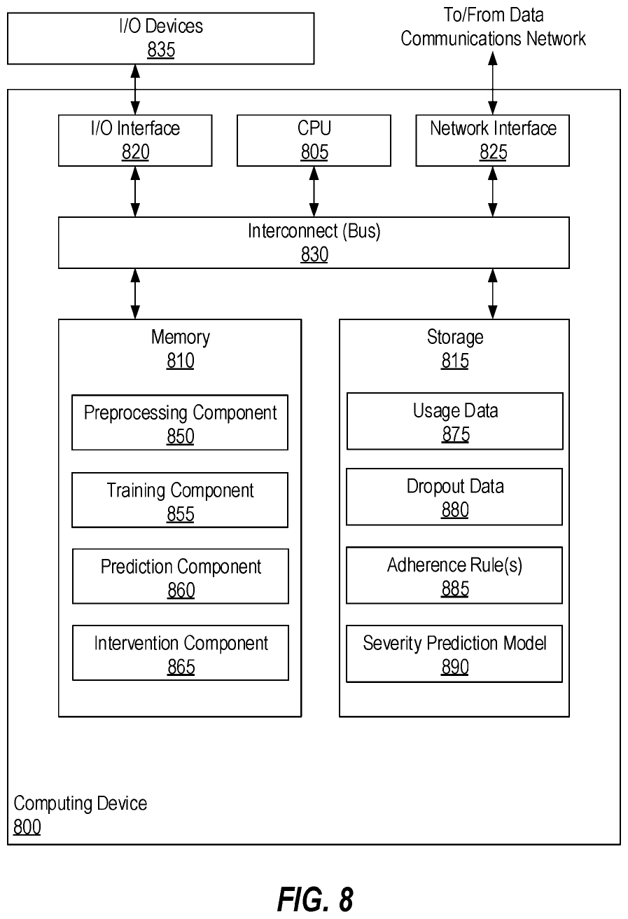

FIG. 6 is a flow diagram depicting an example method 600 for using machine learning to predict dropout severity, according to one embodiment of the present disclosure. In some embodiments, the method 600 is performed by a computing system, such as the training system 125 of FIG. 1, the prediction system 225 of FIG. 2, the intervention system 235 of FIG. 2, and/or the computing device 800 of FIG. 8.

At block 605, first usage information (e.g., usage data 210 of FIG. 2) for a first patient is accessed, the first usage information indicating participation, by the first patient, in a respiratory therapy.

At block 610, a first dropout event with respect to the respiratory therapy is identified based on evaluating the first usage information using a consolidated usage rule (e.g., based on adherence rules 215 of FIG. 2).

At block 615, in response to identifying the first dropout event, a first dropout severity prediction (e.g., predicted severity 230 of FIG. 2) is generated based on the first usage information and a trained machine learning model (e.g., dropout severity model 130 of FIG. 2).

At block 620, in response to determining that the first dropout severity prediction satisfies one or more criteria, one or more interventions (e.g., interventions 240 of FIG. 2) are initiated for the first patient.

### Example Method for Training Machine Learning Models to Predict Dropout Severity

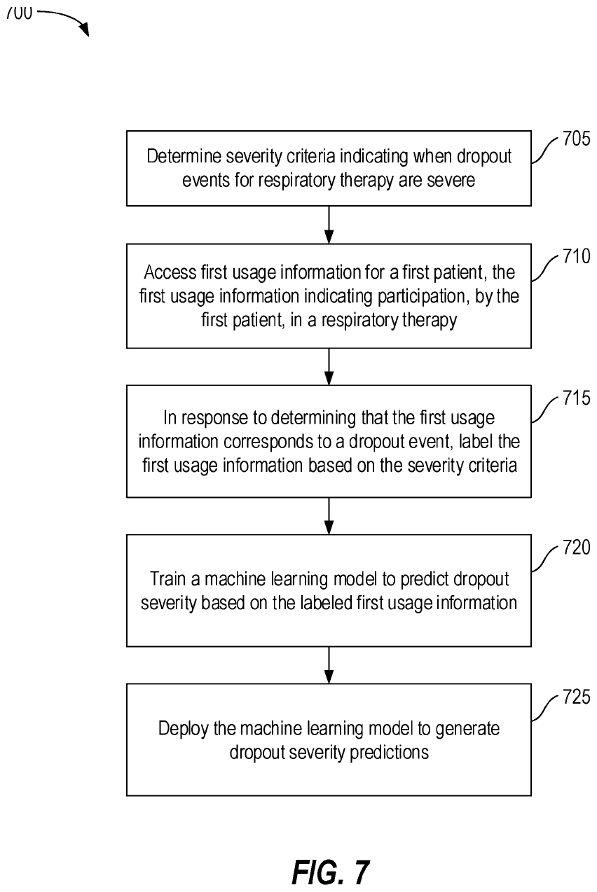

FIG. 7 is a flow diagram depicting an example method 700 for training machine learning models to predict dropout severity, according to one embodiment of the present disclosure. In some embodiments, the method 700 is performed by a computing system, such as the training system 125 of FIG. 1, the prediction system 225 of FIG. 2, the intervention system 235 of FIG. 2, and/or the computing device 800 of FIG. 8.

At block 705, severity criteria indicating when dropout events for respiratory therapy are severe are determined.

At block 710, first usage information (e.g., usage data 110 of FIG. 1) for a first patient is accessed, the first usage information indicating participation, by the first patient, in a respiratory therapy.

At block 715, in response to determining that the first usage information corresponds to a dropout event, the first usage information is labeled based on the severity criteria (e.g., based on dropout records data 120 of FIG. 1).

At block 720, a machine learning model (e.g., dropout severity model 130 of FIG. 1) is trained to predict dropout severity based on the labeled first usage information.

At block 725, the machine learning model is deployed to generate dropout severity predictions.

### Example Computing Device for Dropout Evaluation Using Machine Learning

FIG. 8 depicts an example computing device 800 configured to perform various aspects of the present disclosure. Although depicted as a physical device, in embodiments, the computing device 800 may be implemented using virtual device(s), and/or across a number of devices (e.g., in a cloud environment). In one embodiment, the computing device 800 corresponds to or implements the training system 125 of FIG. 1, the prediction system 225 of FIG. 2, and/or the intervention system 235 of FIG. 2.

As illustrated, the computing device 800 includes a CPU 805, memory 810, a network interface 825, and one or more I/O interfaces 820. Though not included in the depicted example, in some embodiments, the computing device 800 also includes one or more storages. In the illustrated embodiment, the CPU 805 retrieves and executes programming instructions stored in memory 810, as well as stores and retrieves application data residing in memory 810 and/or storage (not depicted). The CPU 805 is generally representative of a single CPU and/or GPU, multiple CPUs and/or GPUs, a single CPU and/or GPU having multiple processing cores, and the like. The memory 810 is generally included to be representative of a random access memory. In an embodiment, if storage is present, it may include any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 835 (such as keyboards, monitors, etc.) are connected via the I/O interface(s) 820. Further, via the network interface 825, the computing device 800 can be communicatively coupled with one or more other devices and components (e.g., via a network, which may include the Internet, local network(s), and the like). As illustrated, the CPU 805, memory 810, network interface(s) 825, and I/O interface(s) 820 are communicatively coupled by one or more buses 830.

In the illustrated embodiment, the memory 810 includes a preprocessing component 850, a training component 855, a prediction component 860, and an intervention component 865, which may perform one or more embodiments discussed above. Although depicted as discrete components for conceptual clarity, in embodiments, the operations of the depicted components (and others not illustrated) may be combined or distributed across any number of components. Further, although depicted as software residing in memory 810, in embodiments, the operations of the depicted components (and others not illustrated) may be implemented using hardware, software, or a combination of hardware and software.

For example, the preprocessing component 850 may be used to perform a variety of preprocessing operations on data used as input to machine learning models, such as filling in missing values, performing scaling on the data, performing feature extraction, and the like. The training component 855 (which may correspond to the training system 125 of FIG. 1) may generally be used to train machine learning models (such as the severity prediction model 890) to predict how severe dropout events will be, as discussed above. The prediction component 860 (which may correspond to the prediction system 225 of FIG. 2) may generally be used to use trained models (such as the severity prediction model 890) to predict patient compliance based on diagnostic data, as discussed above. The intervention component 865 (which may correspond to the intervention system 235 of FIG. 2) may generally be used to evaluate severity predictions to select, perform, and/or facilitate interventions dynamically, as discussed above.

In the illustrated example, the storage 815 includes usage data 875, dropout data 880, adherence rule(s) 885, and severity prediction model 890. Although depicted as residing in storage 815, the depicted components may be stored in any suitable location. The usage data 875 (which may correspond to the usage data 110 and/or usage records 115 of FIG. 1 and/or the usage data 210 of FIG. 2) generally include data relating therapy usage, such as usage durations for one or more days. In some embodiments, the usage data 875 also includes information such as the user's AHI, HI, RERA, and the like. The dropout data 880 (which may correspond to the dropout records 120 of FIG. 1) may generally include information relating to dropout events for patients, such as when the dropout began, whether the dropout was severe, how long the dropout lasted, and the like The adherence rule(s) 885 (which may correspond to adherence rules 215 of FIG. 2) may generally specify criteria used (in various locales or by various entities) to determine whether the patient is compliant with therapy. That is, the adherence rules 885 may generally indicate various ways to define or determine therapy compliance. The severity prediction model 890 (which may correspond to the dropout severity model 130 of FIG. 1 and/or FIG. 2) may be trained and used to generate dropout severity predictions, as discussed above.

### Example Clauses

Clause 1: A method, comprising: accessing first usage information for a first patient, the first usage information indicating participation, by the first patient, in a respiratory therapy; identifying, based on evaluating the first usage information using a consolidated usage rule, a first dropout event with respect to the respiratory therapy; in response to identifying the first dropout event, generating a first dropout severity prediction based on the first usage information and a trained machine learning model; and in response to determining that the first dropout severity prediction satisfies one or more criteria, initiating one or more interventions for the first patient.

Clause 2: The method of Clause 1, further comprising: accessing second usage information for a second patient; identifying, based on evaluating the second usage information using the consolidated usage rule, a second dropout event; in response to identifying the second dropout event, generating a second dropout severity prediction using the trained machine learning model; and in response to determining that the second dropout severity prediction does not satisfy one or more criteria, refraining from initiating one or more interventions for the second patient.

Clause 3: The method of any one of Clauses 1-2, wherein: the first usage information indicates one or more lengths of time that the first patient wore a user interface for the respiratory therapy over one or more days, and generating the first dropout severity prediction comprises: generating a plurality of usage features based on the first usage information; and processing the plurality of usage features using the trained machine learning model.

Clause 4: The method of any one of Clauses 1-3, wherein generating the plurality of usage features comprises extracting features, from the first usage information, corresponding to a defined window of time prior to occurrence of the first dropout event.

Clause 5: The method of any one of Clauses 1-4, further comprising: accessing additional patient information comprising at least one of: (i) an apnea-hypopnea index (AHI) of the first patient; (ii) a hypopnea index (HI) of the first patient; (iii) a number of times the first patient doffed donned the user interface over one or more days; or (iv) a respiratory effort related arousal (RERA) index of the first patient; and extracting one or more features from the additional patient information, wherein generating the first dropout severity prediction further comprises processing the one or more features using the trained machine learning model.

Clause 6: The method of any one of Clauses 1-5, wherein: the consolidated usage rule is defined based on a plurality of adherence rules corresponding to a plurality of locales, and identifying the first dropout event comprises: determining that at least a subset of adherence rules, of the plurality of adherence rules are not satisfied; and determining an earliest time when one of the subset of adherence rules was not satisfied; and determining that the first dropout event occurred at the earliest time.

Clause 7: The method of any one of Clauses 1-6, wherein determining that at least the subset of adherence rules, of the plurality of adherence rules are not satisfied comprises determining that a majority of the plurality of adherence rules are not satisfied.

Clause 8: The method of any one of Clauses 1-7, wherein the first dropout severity prediction indicates whether the first dropout event is predicted to correspond to a non-compliance state that lasts longer than a defined duration.

Clause 9: The method of any one of Clauses 1-8, wherein the trained machine learning model was trained based on a plurality of exemplars, each respective exemplar comprising: a respective set of usage information from a respective period of time prior to a respective dropout event; and a respective label indicating whether the respective dropout event corresponds to non-compliance for longer than the defined duration.

Clause 10: The method of any one of Clauses 1-9, wherein initiating one or more interventions for the first patient comprises at least one of: (i) transmitting a message to the first patient; (ii) providing multimedia content to the first patient; or (iii) notifying one or more healthcare providers associated with the first patient.

Clause 11: A method, comprising: determining severity criteria indicating when dropout events for respiratory therapy are severe; accessing first usage information for a first patient, the first usage information indicating participation, by the first patient, in a respiratory therapy; in response to determining that the first usage information corresponds to a dropout event, labeling the first usage information based on the severity criteria; training a machine learning model to predict dropout severity based on the labeled first usage information; and deploying the machine learning model to generate dropout severity predictions.

Clause 12: The method of Clause 11, wherein: the first usage information indicates one or more lengths of time that the first patient wore a user interface for the respiratory therapy over one or more days, and training the machine learning model comprises: generating a plurality of usage features based on the first usage information, and processing the plurality of usage features using the machine learning model.

Clause 13: The method of any one of Clauses 11-12, wherein generating the plurality of usage features comprises extracting features, from the first usage information, corresponding to a defined window of time prior to occurrence of the dropout event.

Clause 14: The method of any one of Clauses 11-13, further comprising: accessing additional patient information comprising at least one of: (i) an apnea-hypopnea index (AHI) of the first patient; (ii) a hypopnea index (HI) of the first patient; (iii) a number of times the first patient doffed donned the user interface over one or more days; or (iv) a respiratory effort related arousal (RERA) index of the first patient; and extracting one or more features from the additional patient information, wherein training the machine learning model further comprises processing the one or more features using the machine learning model.

Clause 15: The method of any one of Clauses 11-14, wherein: determining that the first usage information corresponds to the dropout event comprises evaluating the first usage information using a consolidated dropout rule, the consolidated usage rule is defined based on a plurality of adherence rules corresponding to a plurality of locales, and identifying the dropout event comprises: determining that at least a subset of adherence rules, of the plurality of adherence rules are not satisfied; and determining an earliest time when one of the subset of adherence rules was not satisfied; and determining that the dropout event occurred at the earliest time.

Clause 16: The method of any one of Clauses 11-15, wherein determining that at least the subset of adherence rules, of the plurality of adherence rules are not satisfied comprises determining that a majority of the plurality of adherence rules are not satisfied.

Clause 17: The method of any one of Clauses 11-16, wherein the severity criteria indicates that dropout events for respiratory therapy are severe if they last longer than a defined duration.

Clause 18: A system, comprising: a memory comprising computer-executable instructions; and one or more processors configured to execute the computer-executable instructions and cause the processing system to perform an operation in accordance with any one of Clauses 1-17.

Clause 19: A system, comprising means for performing a method in accordance with any one of Clauses 1-17.

Clause 20: A non-transitory computer-readable medium comprising computer-executable instructions that, when executed by one or more processors of a processing system, cause the processing system to perform a method in accordance with any one of Clauses 1-17.

Clause 21: A computer program product embodied on a computer-readable storage medium comprising code for performing a method in accordance with any one of Clauses 1-17.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications (e.g., the computing device 800) or related data available in the cloud. For example, the computing device 800 could execute on a computing system in the cloud and train and/or use machine learning models to predict short-term compliance with respiratory therapy and/or assign users to setup classes. In such a case, the computing device 800 could train machine learning models, and store the trained models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing first usage information for a first patient, the first usage information indicating participation, by the first patient, in a respiratory therapy;
identifying, based on evaluating the first usage information using a consolidated usage rule, a first dropout event with respect to the respiratory therapy;
in response to identifying the first dropout event, generating a first dropout severity prediction based on the first usage information and a trained machine learning model; and
in response to determining that the first dropout severity prediction satisfies one or more criteria, initiating one or more interventions for the first patient.

2. The method of claim 1, further comprising:
accessing second usage information for a second patient;
identifying, based on evaluating the second usage information using the consolidated usage rule, a second dropout event;
in response to identifying the second dropout event, generating a second dropout severity prediction using the trained machine learning model; and
in response to determining that the second dropout severity prediction does not satisfy one or more criteria, refraining from initiating one or more interventions for the second patient.

3. The method of claim 1, wherein:
the first usage information indicates one or more lengths of time that the first patient wore a user interface for the respiratory therapy over one or more days, and
generating the first dropout severity prediction comprises:
generating a plurality of usage features based on the first usage information; and
processing the plurality of usage features using the trained machine learning model.

4. The method of claim 3, wherein generating the plurality of usage features comprises extracting features, from the first usage information, corresponding to a defined window of time prior to occurrence of the first dropout event.

5. The method of claim 3, further comprising:
accessing additional patient information comprising at least one of:
(i) an apnea-hypopnea index (AHI) of the first patient;
(ii) a hypopnea index (HI) of the first patient;
(iii) a number of times the first patient doffed donned the user interface over one or more days; or
(iv) a respiratory effort related arousal (RERA) index of the first patient; and
extracting one or more features from the additional patient information, wherein generating the first dropout severity prediction further comprises processing the one or more features using the trained machine learning model.

6. The method of claim 1, wherein:
the consolidated usage rule is defined based on a plurality of adherence rules corresponding to a plurality of locales, and
identifying the first dropout event comprises:
determining that at least a subset of adherence rules, of the plurality of adherence rules are not satisfied; and
determining an earliest time when one of the subset of adherence rules was not satisfied; and
determining that the first dropout event occurred at the earliest time.

7. The method of claim 6, wherein determining that at least the subset of adherence rules, of the plurality of adherence rules are not satisfied comprises determining that a majority of the plurality of adherence rules are not satisfied.

8. The method of claim 1, wherein the first dropout severity prediction indicates whether the first dropout event is predicted to correspond to a non-compliance state that lasts longer than a defined duration.

9. The method of claim 8, wherein the trained machine learning model was trained based on a plurality of exemplars, each respective exemplar comprising:
a respective set of usage information from a respective period of time prior to a respective dropout event; and
a respective label indicating whether the respective dropout event corresponds to non-compliance for longer than the defined duration.

10. The method of claim 1, wherein initiating one or more interventions for the first patient comprises at least one of:
(i) transmitting a message to the first patient;
(ii) providing multimedia content to the first patient; or
(iii) notifying one or more healthcare providers associated with the first patient.

11. A method, comprising:
determining severity criteria indicating when dropout events for respiratory therapy are severe;
accessing first usage information for a first patient, the first usage information indicating participation, by the first patient, in a respiratory therapy;
in response to determining that the first usage information corresponds to a dropout event, labeling the first usage information based on the severity criteria;
training a machine learning model to predict dropout severity based on the labeled first usage information; and
deploying the machine learning model to generate dropout severity predictions.

12. The method of claim 11, wherein:
the first usage information indicates one or more lengths of time that the first patient wore a user interface for the respiratory therapy over one or more days, and
training the machine learning model comprises:
generating a plurality of usage features based on the first usage information; and
processing the plurality of usage features using the machine learning model.

13. The method of claim 12, wherein generating the plurality of usage features comprises extracting features, from the first usage information, corresponding to a defined window of time prior to occurrence of the dropout event.

14. The method of claim 13, further comprising:
accessing additional patient information comprising at least one of:
(i) an apnea-hypopnea index (AHI) of the first patient;
(ii) a hypopnea index (HI) of the first patient;
(iii) a number of times the first patient doffed donned the user interface over one or more days; or
(iv) a respiratory effort related arousal (RERA) index of the first patient; and
extracting one or more features from the additional patient information, wherein training the machine learning model further comprises processing the one or more features using the machine learning model.

15. The method of claim 11, wherein:
determining that the first usage information corresponds to the dropout event comprises evaluating the first usage information using a consolidated dropout rule,
the consolidated usage rule is defined based on a plurality of adherence rules corresponding to a plurality of locales, and
identifying the dropout event comprises:
determining that at least a subset of adherence rules, of the plurality of adherence rules are not satisfied; and
determining an earliest time when one of the subset of adherence rules was not satisfied; and
determining that the dropout event occurred at the earliest time.

16. The method of claim 15, wherein determining that at least the subset of adherence rules, of the plurality of adherence rules are not satisfied comprises determining that a majority of the plurality of adherence rules are not satisfied.

17. The method of claim 11, wherein the severity criteria indicates that dropout events for respiratory therapy are severe if they last longer than a defined duration.

18. A non-transitory computer-readable medium comprising computer-executable instructions that, when executed by one or more processors of a processing system, cause the processing system to perform an operation comprising:
accessing first usage information for a first patient, the first usage information indicating participation, by the first patient, in a respiratory therapy;
identifying, based on evaluating the first usage information using a consolidated usage rule, a first dropout event with respect to the respiratory therapy;
in response to identifying the first dropout event, generating a first dropout severity prediction based on the first usage information and a trained machine learning model; and
in response to determining that the first dropout severity prediction satisfies one or more criteria, initiating one or more interventions for the first patient.

19. The non-transitory computer-readable medium of claim 18, the operation further comprising:
accessing second usage information for a second patient;
identifying, based on evaluating the second usage information using the consolidated usage rule, a second dropout event;
in response to identifying the second dropout event, generating a second dropout severity prediction using the trained machine learning model; and
in response to determining that the second dropout severity prediction does not satisfy one or more criteria, refraining from initiating one or more interventions for the second patient.

20. The non-transitory computer-readable medium of claim 18, wherein:
the consolidated usage rule is defined based on a plurality of adherence rules corresponding to a plurality of locales, and
identifying the first dropout event comprises:
determining that at least a subset of adherence rules, of the plurality of adherence rules are not satisfied; and
determining an earliest time when one of the subset of adherence rules was not satisfied; and
determining that the first dropout event occurred at the earliest time.
